(19) United States
(12) Patent Application Publication
(10) Pub. No.: US 2022/0046152 A1
(43) Pub. Date: Feb. 10, 2022

(54) PREVENTION OF INFORMATION EXPOSURE TO A LABORATORY INSTRUMENT

(71) Applicant: Beckman Coulter, Inc., Brea, CA (US)

(72) Inventor: John HETZLER, Victoria, MN (US)

(21) Appl. No.: 17/507,902

(22) Filed: Oct. 22, 2021

Related U.S. Application Data
(63) Continuation of application No. PCT/US20/29497, filed on Apr. 23, 2020.
(60) Provisional application No. 62/837,437, filed on Apr. 23, 2019.

(51) Int. Cl. H04N 5/225; H04N 7/18; G16H 10/40; G06F 21/60

(52) U.S. Cl. CPC H04N 5/2254; G06F 21/60; G16H 10/40; H04N 7/18

(57) ABSTRACT

Disclosed subject matter relates to laboratory instrument to prevent information exposure from at least one image capturing device coupled to the laboratory instrument, that captures at least one of images and videos during operations of the laboratory instrument, a transparent member coupled to the laboratory instrument, that enables an internal view into the laboratory instrument from outside the laboratory instrument, and one or more optical means coupled to the transparent member in a predefined manner that obstructs a Field of View (FOV) of the at least one image capturing device beyond the transparent member and prevents exposure of any external information present proximate to the laboratory instrument, which helps in maintaining data privacy.

### PRIORITY

This is related to, and claims the benefit of, previously filed provisional application 62/837,437, filed in the United States on Apr. 23, 2019 and titled prevention of information exposure to a laboratory instrument. The disclosure of that application is hereby incorporated by reference in its entirety.

### TECHNICAL FIELD

This disclosure relates generally to diagnostic instruments.

### BACKGROUND

Generally, laboratory instruments, and specifically diagnostic instruments, may be associated with one or more devices to perform various operations of the instruments. Usually, in a few laboratory instruments there may be a transparent sheet that allows users in a laboratory to view operations of the instruments. Usually, such a transparent sheet around the laboratory instruments may also protect the users from potential hazards that may occur due to moving components associated with the instruments. Normally, various operations of the instruments may be captured in the form of images and/or videos by an image capturing device, such as a camera. The image capturing device may be configured in the instrument or the instrument may be externally associated with the instrument.

However, due to the presence of transparent sheet around the instrument, surrounding environment in the laboratory may be visible to the image capturing device. Usually, due to this type of arrangement, the image capturing device may capture images and/or videos of objects that are proximate to the instruments, which may be beyond the transparent sheet, along with the images and/or videos of operations of the instruments. Usually, capturing images and/or videos of objects beyond the transparent sheet may include a risk of exposing information that may be confidential in nature, such as personal information, that may be present proximate to the instrument, thereby compromising on data privacy.

The present disclosure provides an instrument with means to prevent exposure of external information proximate to the instrument within the laboratory, thereby ameliorating the current disadvantage.

The information disclosed in this background of the disclosure section is only for enhancement of understanding of the general background of the disclosure and should not be taken as an acknowledgement or any form of suggestion that this information forms prior art already known to a person skilled in the art.

### SUMMARY

One or more shortcomings of the prior art may be overcome, and additional advantages may be provided through embodiments of the present disclosure. Additional features and advantages may be realized through the techniques of the present disclosure. Other embodiments and aspects of the disclosure are described in detail herein and are considered a part of the claimed disclosure.

Embodiments of the present disclosure relate to a laboratory instrument, and more specifically to a diagnostic instrument. In one embodiment, the instrument (hereinafter referred to as a laboratory instrument or a diagnostic instrument) includes at least one image capturing device configured to capture at least one still image or video during operation of the instrument. In a further embodiment, the instrument includes a transparent member coupled to the instrument. In a further embodiment, the transparent member enables an internal view of the instrument from outside the instrument. In a further embodiment, the instrument includes one or more optical resources coupled to the transparent member in a predefined or preconfigured manner. In a further embodiment, the one or more optical resources obstruct a Field of View (FOV) of the at least one image capturing device beyond the transparent member to prevent exposure of any external information present proximate to the instrument.

The foregoing summary is only illustrative in nature and is not intended to be in any way limiting on the embodiments disclosed herein. In addition to the illustrative aspects, embodiments, and features described above, further aspects, embodiments, and features will become apparent by reference to the drawings and the following detailed description.

### BRIEF DESCRIPTION OF THE ACCOMPANYING DIAGRAMS

The accompanying drawings, which are incorporated in and constitute a part of this disclosure, illustrate exemplary embodiments and, together with the description, serve to explain the disclosed principles. In the figures, the left-most digit(s) of a reference number identifies the figure in which the reference number first appears. The same reference numbers are used throughout the figures to reference like features and components. Some embodiments of system and/or methods in accordance with embodiments of the present subject matter are now described, by way of example only, and with reference to the accompanying figures, in which:

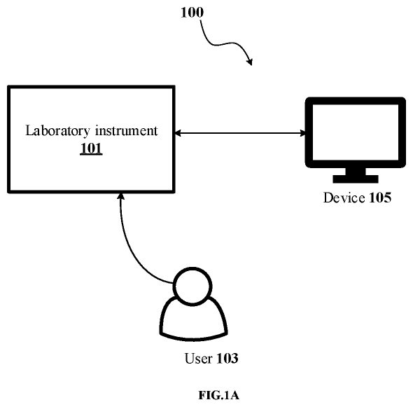

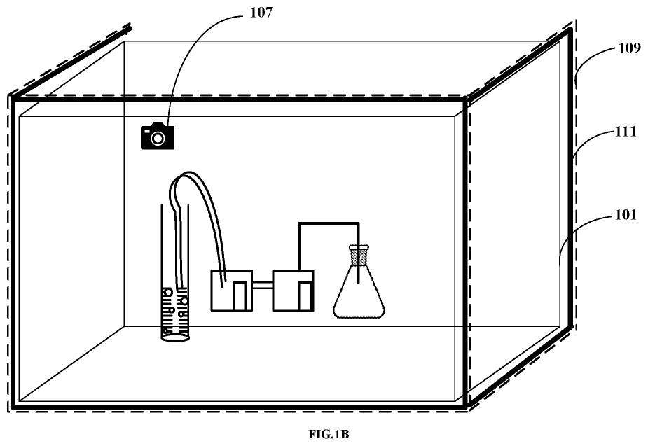

FIG. 1A illustrates an exemplary architecture to prevent exposure of external information present proximate to a laboratory (diagnostic) instruments in accordance with embodiments of the present disclosure;

FIG. 1B illustrate an exemplary block diagram of a laboratory instrument in accordance with embodiments of the present disclosure;

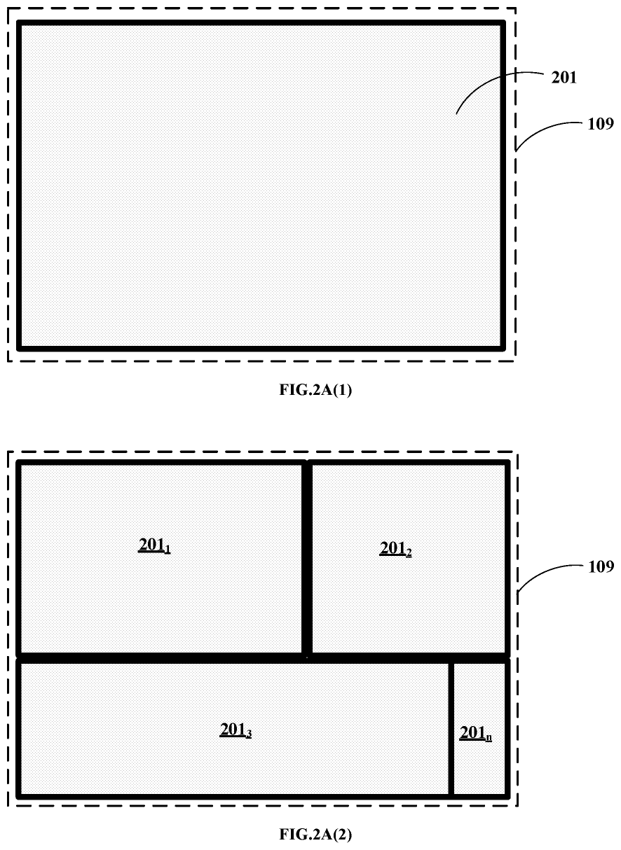

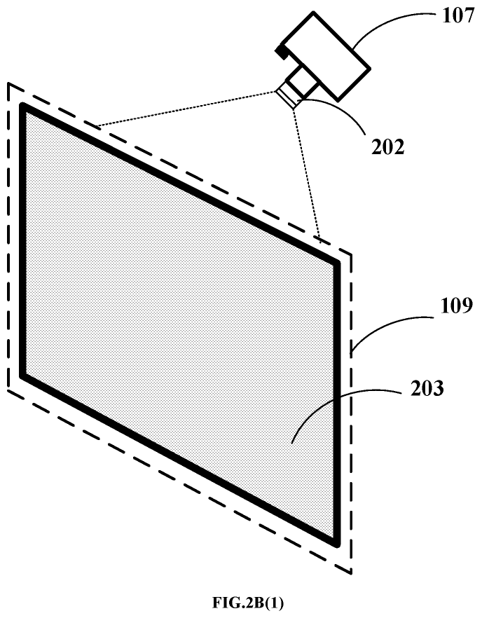

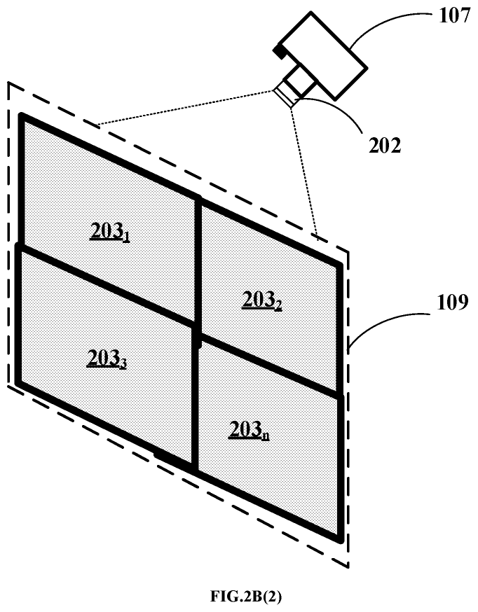

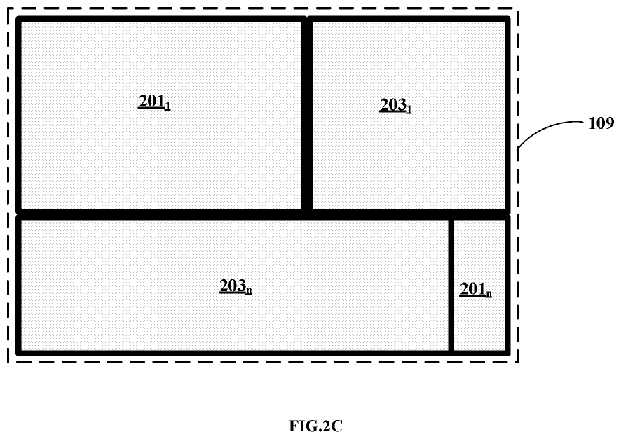

FIG. 2A(1) and FIG. 2A(2) illustrate exemplary coupling of a one-way mirror film to a transparent member in accordance with an embodiment of the present disclosure;

FIG. 2B(1) and FIG. 2B(2) illustrate exemplary coupling of a polarizing material to a transparent member in accordance with an alternate embodiment of the present disclosure;

FIG. 2C illustrates exemplary coupling of a one-way mirror film and a polarizing material to transparent member in accordance with an embodiment of the present disclosure; and

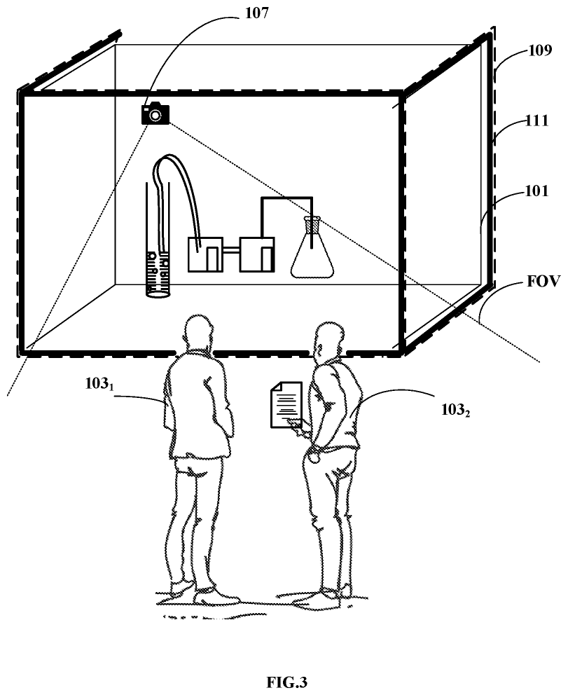

FIG. 3 illustrates an exemplary laboratory setup in accordance with an embodiment of the present disclosure.

It should be appreciated by those skilled in the art that any block diagrams herein represent conceptual views of illustrative systems embodying the principles of the present subject matter. Similarly, it will be appreciated that any flow charts, flow diagrams, state transition diagrams, pseudo code, and the like represent various processes which may be substantially represented in computer readable medium and executed by a computer or processor, whether or not such computer or processor is explicitly shown.

### DETAILED DESCRIPTION

In the following detailed description of the embodiments of the disclosure, reference is made to the accompanying drawings that form a part hereof, and in which are shown by way of illustration specific embodiments in which the disclosure may be practiced. These embodiments are described in sufficient detail to enable those skilled in the art to practice the teachings of this disclosure, and it is to be understood that other embodiments may be utilized and that changes may be made without departing from the scope of the present disclosure. The following description is, therefore, not to be taken in a limiting sense. A description of an embodiment with several components in communication with each other does not imply that all such components are required. On the contrary a variety of optional components are described to illustrate the wide variety of possible embodiments of the invention.

In the present document, the word “exemplary” is used herein to mean “serving as an example, instance, or illustration.” Any embodiment or implementation of the present subject matter described herein as “exemplary” is not necessarily construed to be as preferred or advantageous over other embodiments that may be disclosed.

While the disclosure is susceptible to various modifications and alternative forms, specific embodiments thereof have been illustrated by way of example in the drawings and will be described in detail below. It should be understood, however that this is not intended to limit the disclosure to the forms disclosed, but on the contrary, the disclosure is to cover all modifications, equivalents, and alternative falling within the scope of the disclosure.

The terms “comprises”, “comprising”, “includes” or any other variations thereof, are intended to cover a non-exclusive inclusion, such that a setup, device or method that includes a list of components or steps does not include only those components or steps but may include other components or steps not expressly listed or inherent to such setup or device or method. In other words, one or more elements in a system or apparatus preceeded by “comprises . . . a” does not, without more constraints, preclude the existence of other elements or additional elements in the system or method. Also, the words “comprising,” “having,” “containing,” and “including,” and other similar forms are intended to be equivalent in meaning and be open-ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

According to one embodiment, a method of preventing information exposure to an image capturing device associated with a laboratory device may be provided. Such a method may comprise the image capturing device capturing at least one still image or video during operation of the laboratory device. Such a method may comprise preventing exposure of external information proximate to the laboratory instrument by obstructing a field of view of the image capturing device beyond a transparent member. In such a method, the transparent member may enable an internal view of the laboratory device from outside of the laboratory device.

According to a further embodiment, in some methods such as described in the preceding paragraph, the field of view of the image capturing device beyond the transparent member may be obstructed in real time.

According to a further embodiment, in some methods such as described in any of the preceding two paragraphs a user may define the field of view.

According to a further embodiment, in some methods such as described in any of the preceding three paragraphs, the field of view of the image capturing device may be obstructed by one or more optical resources comprising a one-way mirror coupled to an inner wall of the transparent member. According to a further embodiment, in some methods such as described in any of the preceding three paragraphs, the field of view of the image capturing device may be obstructed by one or more optical resources comprising a polarizing material coupled to an inner wall of the transparent member. In some such embodiments, the image capturing device may be coupled with a polarizing surface.

According to a further embodiment, in some methods such as described in any of the preceding four paragraphs, the image capturing device may comprise a camera.

According to a further embodiment, in some methods such as described in any of the preceding five paragraphs, the eternal information may comprise any object beyond the transparent member.

According to a further embodiment, in some methods such as described in any of the preceding six paragraphs, the laboratory device may be a laboratory instrument.

According to a further embodiment, there may be provided a laboratory device which, in some embodiments may be a laboratory instrument. In some embodiments, such a laboratory device may comprise at least one image capturing device associated with the laboratory device, to capture at least one still image or video during operation of the laboratory device; a transparent member coupled to the laboratory device, enabling an internal view into the laboratory device from outside the laboratory device; and one or more optical resources coupled to the transparent member for obstructing a Field of View (FOV) of the at least one image capturing device beyond the transparent member preventing exposure of any external information proximate to the laboratory device.

According to a further embodiment, a device such as described in the context of the preceding paragraph may be provided in which the one or more optical resources may comprise a one-way mirror coupled to an inner wall of the transparent member.

According to a further embodiment, a device such as described in the context of any of the preceding two paragraphs may be provided in which, the one or more optical resources may comprise a polarizing material coupled to an inner wall of the transparent member. In some such embodiments, the image capturing device may be coupled with a polarizing interface.

According to a further embodiment, a device such as described in the context of any of the preceding three paragraphs may be provided in which the image capturing device may comprise a camera.

According to a further embodiment, a device such as described in the context of any of the preceding four paragraphs may be provided in which the external information may comprise any object beyond the transparent member.

According to a further embodiment, a system may be provided that comprises a laboratory device as described in any of the preceding five paragraphs coupled to a computing device configured to store and/or display images captured by the image capturing device.

Embodiments disclosed herein may include a laboratory instrument (also generally referred to as a diagnostic instrument, and is interchangeably used). Usually, the laboratory instrument is a diagnostic instrument. In one embodiment, the laboratory instrument may also include a non-diagnostic instrument. In a further embodiment, the laboratory instrument may include other healthcare related instruments. The laboratory instrument may be associated or coupled with one or more devices to perform at least one operation of the laboratory instrument. In some embodiments, at least one image capturing device may be coupled to the laboratory instrument. In some embodiments, the phrase “at least one image capturing device” and the word “image capturing device/s” may be alternatively used. In some embodiments, the image capturing device(s) may be configured to capture at least one image or video proximate to the laboratory instrument, during operations of the laboratory instrument. In some embodiments, the image capturing device(s) may be stationary having a fixed Field of View (FOV). In some other embodiments, the image capturing device(s) may be movable having a varying FOV. In some embodiments, a transparent member may be coupled to the laboratory instrument. In some embodiments, the transparent member may completely enclose the laboratory instrument. Further, in some other embodiments, the transparent member may cover at least one portion of the laboratory instrument. In certain other embodiments, the transparent member may be configured to enable an internal view into the laboratory instrument from outside the laboratory instrument.

In some embodiments, one or more optical resources may be coupled to the transparent member. In some embodiments, the phrase “one or more optical resources” and the word “optical resource/s” may be alternatively used. In some other embodiments, the optical resources may be configured in a predefined manner. In yet some other embodiments, the optical resource may be a one-way minor formed by a coating of one-way mirror film. Further, in some other embodiments, the optical resource may be a polarizing material. In yet some other embodiments, the optical resource may be a combination of the one-way mirror and the polarizing material. In some embodiments, the image capturing device(s) may be coupled with a polarizing surface when the optical resource is the polarizing material. In some embodiments, the polarizing surface may be a polarizing lens or any other means capable of attaining polarizing effect. In some other embodiments, the optical resources may be configured to obstruct a Field of View (FOV) of the image capturing device(s) beyond the transparent member to prevent exposure of external information (also referred to broadly as content or data) proximate to the laboratory instrument. In yet some other embodiments, any element/object existing within the FOV of the image capturing device(s) beyond the transparent member of the laboratory instrument may be considered as proximate to the laboratory instrument. In some other embodiments, the external information may be any object such as information, data, content, humans, pictures, documents and the like, existing beyond the transparent member.

In some embodiments, the laboratory instrument may be associated with a device to display and store the one or more images captured by the image capturing device(s).

In the present disclosure the optical resources coupled to the transparent member help in obstructing the FOV of the image capturing device(s) beyond the transparent member. This kind of obstruction prevents exposure of any external information existing proximate to the laboratory instrument, without causing any obstruction for a user standing beyond the transparent member, to view the laboratory instrument.

Reference is now made to FIG. 1A, which illustrates an exemplary architecture to prevent exposure of external information existing proximate to the laboratory instruments in accordance with embodiments of the present disclosure.

System architecture 100 includes laboratory instrument 101, user 103 and device 105, having at least a display unit. In an embodiment, laboratory instrument 101 may be a diagnostic instrument. In another embodiment, laboratory instrument 101 may be a non-diagnostic instrument. In a further embodiment, laboratory instrument 101 may be any other healthcare related instrument. In one embodiment, laboratory instrument 101 may be associated with device 105 via a communication network (not shown in the FIG. 1A). The communication network is at least one of a wired communication network or a wireless communication network or a combination thereof. As an example, device 105 may include, but may not be limited to, a mobile phone, a laptop computer, a desktop computer and a tablet. Device 105 may be locally or remotely associated with laboratory instrument 101. In some embodiments, user 103 may be a laboratory technician, a visitor or any other person viewing laboratory instrument 101 or present proximate to laboratory instrument 101.

Reference is now made to FIG. 1B, which illustrates a block diagram of laboratory instrument 101, which is coupled with at least one image capturing device 107 (also referred as image capturing device(s) 107), transparent member 109 and one or more optical resources 111 (also referred as optical resource/s 111).

Laboratory instrument 101 is associated with one or more devices (not shown in FIG. 1B) to perform operations of laboratory instrument 101. The one or more devices are interconnected to interactively perform operations of laboratory instrument 101.

Image capturing device 107 is configured to capture at least one image or video during operations of laboratory instrument 101. As an example, image capturing device 107 may include, but may not be limited to, a camera. As an example, the camera may be a monochrome camera, a color camera, digital camera and the like, of desired resolution, that may be configured to capture still images periodically or videos continuously during the operation of laboratory instrument 101. Further, image capturing device 107 may transmit at least one of the images and/or the videos to device 105. Device 105 is configured to store the images and videos received in a storage unit associated with device 105 and also configured to display the images and videos to user 103 based on requirement of user 103. In an exemplary embodiment, the storage unit may be associated with device 105 locally or remotely. In an exemplary embodiment, the storage unit may be configured within the device 105.

Further, transparent member 109 encloses laboratory instrument 101, which enables an external user of an internal view into laboratory instrument 101 from outside laboratory instrument 101. Further, transparent member 109 protects user 103 from potential hazards that may occur due to at least one of, movement of the one or more components, reagents used by laboratory instrument 101 and the like. As an example, transparent member 109 may be a transparent glass, transparent sheet made of material such as plastic and the like, transparent LCD panels, transparent ceramic and the like.

One or more optical resources 111 is coupled to transparent member 109 of laboratory instrument 101. In one embodiment, optical resource 111 is a one-way mirror. A one-way mirror is formed by coating a thin layer of metal, for example a light metal such as Aluminum and the like, on transparent member 109. The thin layer is referred as one-way minor film. This coating enables transparent member 109 to attain properties of a one-way mirror that reflects some light and allows rest of the light to penetrate through it. The one way mirror film is placed on an inner wall of transparent member 109 in a predefined or preconfigured manner. In an embodiment, inner wall is only illustrative and the disclosure can be extended to inner walls (more than one inner wall) of transparent member 109. The predefined manner includes completely covering inner walls of transparent member 109. In an exemplary embodiment, single one-way mirror film 201 is used on each side of transparent member 109 as shown in FIG. 2A(1) to completely cover transparent member 109. In a further exemplary embodiment, multiple one-way minor films 201₁ to 201ₙ may be used on each side of transparent member 109 as shown in FIG. 2A(2) to completely cover transparent member 109.

The one-way mirror works effectively when there is a difference in intensity of light on either side of the one-way minor. More precisely, the one-way minor acquires reflective properties of a mirror when one side of the one-way mirror faces light of high intensity (also referred as “bright side” or “reflective side”) compared to other side of the one-way minor which faces light of low intensity i.e. a relatively dim condition (also referred as “dark side” or “non-reflective side”). The bright side of the one-way minor reflects light, whereas the dark side of the one-way mirror allows penetration of the light. In the context of the present disclosure, the one-way mirror works effectively when a region within transparent member 109 is brightly lit compared to region beyond transparent member 109.

Therefore, in the present disclosure, the reflective side of the one-way minor faces image capturing device 107 and the non-reflective side of the one-way minor faces away from image capturing device 107 i.e. towards the region beyond transparent member 109. When image capturing device(s) 107 captures images and videos during the operations of laboratory instrument 101, the reflective side of the one-way mirror obstructs a Field of View (FOV) of image capturing device(s) 107 beyond transparent member 109, by showcasing reflection of a region within transparent member 109. This obstruction of FOV using reflective property of the one-way minor prevents exposure of any external information proximate to laboratory instrument 101. In an exemplary embodiment, the external information may be any information beyond transparent member 109 present in the at least one of images and videos. As an example, the external information captured in the at least one image or video may include, but not limited to, Personally Identifiable Information (PII), users proximate to laboratory instrument 101, documents carried by users, other laboratory instruments situated beyond transparent member 109 and any other element that does not contribute in performing the operations of laboratory instrument 101.

Further, in an alternative embodiment, optical resource 111 may be a polarizing material. The polarizing material may include, but not limited to, a synthetic plastic sheet. The polarizing material behaves as an optical filter that may allow light waves of only specific polarization to penetrate. In some embodiments, the polarizing material may be used in combination with a polarizing surface. In some embodiments, the polarizing surface may include, but not limited to, a polarizing lens. In an exemplary embodiment, polarizing surface 202 may be coupled to image capturing device(s) 107 as shown in the FIG. 2B(1). In one embodiment, polarizing surface 202 may be mounted over existing lens configured in image capturing device(s) 107. In some embodiments, the polarizing material may be coupled to the inner wall of transparent member 109 in a predefined manner. In an embodiment, inner wall is only illustrative and the disclosure can be extended to inner walls (more than one inner wall) of transparent member 109. The predefined manner may include coupling the polarizing material to the inner walls in a way that polarizing surface 202 and the polarizing material may be out of phase or in other words, out of sync. Further, the predefined manner may also include completely covering the inner walls of transparent member 109. In an exemplary embodiment, single polarizing material 203 may be used on each side of transparent member 109 as shown in FIG. 2B(1) to completely cover transparent member 109. In a further exemplary embodiment, multiple polarizing materials 203₁ to 203ₙ may be used on each side of transparent member 109 as shown in FIG. 2B(2) to completely cover transparent member 109.

In some embodiments, when image capturing device(s) 107 begins to capture the images or videos via polarizing surface 202, the polarizing material which is out of phase with polarizing surface 202 may create a blur effect or a distortion that may obstruct the FOV of image capturing device(s) 107 beyond transparent member 109, thereby preventing exposure of the external information present in proximate to laboratory instrument 101.

In an alternate embodiment, optical resources 111 may be a combination of the one-way mirror and the polarizing material as shown in the FIG. 2C.

Reference is now made to FIG. 3 that shows an exemplary laboratory setup where user 103₁ and user 103₂ are viewing the operations of laboratory instrument 101 in close proximity to laboratory instrument 101, but beyond transparent member 109. Consider user 103₁ is wearing an identity card that include personal details of user 103₁. Further, consider user 103₂ is holding a document having confidential information. When image capturing device 107 captures the images during the operations of laboratory instrument 101, image capturing device 107 may also capture the external information beyond transparent member 109 that is present within the FOV of image capturing device 107. In this scenario, the external information may be user 103₁, user 103₂, identity card of user 103₁ and document held by user 103₂. However, when transparent member 109 is coupled with the optical resources such as the one-way mirror or the polarizing material, the FOV of image capturing device 107 can be obstructed beyond the transparent member 109 such that, the images and/or videos captured by image capturing device 107 includes only operations of laboratory instrument 101.

In an embodiment, the present disclosure provides a laboratory instrument for prevention of information exposure to a laboratory instrument. In a further embodiment, the optical resources coupled to the transparent member obstruct the FOV of the image capturing device(s) beyond the transparent member. This kind of obstruction prevents exposure of any external information present proximate to the laboratory instrument, without causing any obstruction for a user standing beyond the transparent member, to view the laboratory instrument. Further, when the optical resource is a one-way mirror, the optical resource not only obstructs the FOV of the image capturing device(s), but enables the image capturing device to capture reflection of the laboratory instrument observed on the reflective side of the one-way mirror. This feature of the present disclosure enables users to achieve a view of opposite side of the laboratory instrument.

As described herein a description of an embodiment with several components in communication with each other does not imply that all such components are required. On the contrary a variety of optional components are described to illustrate the wide variety of possible embodiments of the invention.

When a single device or article is described herein, it will be apparent that more than one device/article (whether or not they cooperate) may be used in place of a single device/article. Similarly, where more than one device or article is described herein (whether or not they cooperate), it will be apparent that a single device/article may be used in place of the more than one device or article or a different number of devices/articles may be used instead of the shown number of devices or programs. The functionality and/or the features of a device may be alternatively embodied by one or more other devices which are not explicitly described as having such functionality/features. Thus, other embodiments of the disclosure need not include the device itself.

The specification describes a laboratory instrument for prevention of information exposure to a laboratory instrument. The illustrated steps are set out to explain exemplary embodiments shown, and it should be anticipated that on-going technological development will change the manner in which particular functions are performed. These examples are presented herein for purposes of illustration, and not as a limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope and spirit of the disclosed embodiments.

Finally, the language used in the specification has been principally selected for readability and instructional purposes, and it may not have been selected to delineate or circumscribe the inventive subject matter. It is therefore intended that the scope of the invention be limited not by this detailed description, but rather by any claims that issue on an application based here on. Accordingly, the embodiments of the present disclosure are intended to be illustrative, but not limiting, of the scope of the disclosure, which is set forth in the following claims.

1. A method of preventing information exposure to an image capturing device associated with a laboratory device, the method comprising:
(a) the image capturing device capturing at least one still image or video during operation of the laboratory device; and
(b) preventing exposure of external information proximate to the laboratory device by obstructing a field of view of the image capturing device beyond a transparent member;
wherein the transparent member enables an internal view of the laboratory device from outside of the laboratory device.

2. The method of claim 1, wherein the field of view of the image capturing device beyond the transparent member is obstructed in real time.

3. The method of claim 2, wherein a user defines the field of view.

4. The method of claim 1, wherein the field of view of the image capturing device is obstructed by one or more optical resources comprising a one-way mirror coupled to an inner wall of the transparent member.

5. The method of claim 1, wherein the field of view of the image capturing device is obstructed by one or more optical resources comprising a polarizing material coupled to an inner wall of the transparent member.

6. The method of claim 5, wherein the image capturing device is coupled with a polarizing surface.

7. The method of claim 1, wherein the image capturing device comprises a camera.

8. The method of claim 1, wherein the external information comprises any object beyond the transparent member.

9. A laboratory device comprising:
(a) at least one image capturing device associated with the laboratory device, to capture at least one still image or video during operation of the laboratory device;
(b) a transparent member coupled to the laboratory device, enabling an internal view into the laboratory device from outside the laboratory device; and
(c) one or more optical resources coupled to the transparent member and adapted to obstruct a field of view (FOV) of the at least one image capturing device beyond the transparent member preventing exposure of any external information proximate to the laboratory device.

10. The laboratory device of claim 9, wherein the one or more optical resources comprises a one-way mirror coupled to an inner wall of the transparent member.

11. The laboratory device of claim 9, wherein the one or more optical resources comprises a polarizing material coupled to an inner wall of the transparent member.

12. The laboratory device of claim 11, wherein at least one image capturing device is coupled with a polarizing surface.

13. The laboratory device of claim 9, wherein the image capturing device comprises a camera.

14. The laboratory device of claim 9, wherein the external information comprises any object beyond the transparent member.

15. A system comprising:
(a) a laboratory device comprising:
(i) at least one image capturing device associated with the laboratory device, to capture at least one still image or video during operation of the laboratory device;
(ii) a transparent member coupled to the laboratory device, enabling an internal view into the laboratory device from outside the laboratory device; and
(iii) one or more optical resources coupled to the transparent member and adapted to obstruct a field of view (FOV) of the at least one image capturing device beyond the transparent member preventing exposure of any external information proximate to the laboratory device;
and
(b) a computing device configured to store and display images captured by the image capturing device, wherein the laboratory device is coupled to the computing device.

16. The system claim 15, wherein the one or more optical resources comprises a one-way mirror coupled to an inner wall of the transparent member.

17. The system of claim 15, wherein the one or more optical resources comprises a polarizing material coupled to an inner wall of the transparent member.

18. The system of claim 17, wherein at least one image capturing device is coupled with a polarizing surface.

19. The system of claim 15, wherein the image capturing device comprises a camera.

20. The system of claim 15, wherein the external information comprises any object beyond the transparent member.
